(19) United States
(12) Patent Application Publication
(10) Pub. No.: US 2013/0197581 A1
(43) Pub. Date: Aug. 1, 2013

(54) SPINOUS PROCESS IMPLANT WITH TEMPORARILY EXTENDED POST

(71) Applicants: Jeff R. Justis, Germantown, TN (US); Gregory C. Marik, Collierville, TN (US); Charles S. Sullivan, Germantown, TN (US)

(72) Inventors: Jeff R. Justis, Germantown, TN (US); Gregory C. Marik, Collierville, TN (US); Charles S. Sullivan, Germantown, TN (US)

(73) Assignee: Warsaw Orthopedic, Inc., Warsaw, IN (US)

(21) Appl. No.: 13/361,497

(22) Filed: Jan. 30, 2012

(51) Int. Cl. A61B 17/70; A61B 17/88

(52) U.S. Cl. 606/248; 606/279

(57) ABSTRACT

Spinous process implants and methods relate to a spinal implant with two plates that are connected together by a post. The implant is configured for each plate to be positioned on outer lateral sides of spinous processes with the post extending through the interspinous space. The second of the two plates includes a bore that receives the post, and that plate is movable along the length of the post, and selectively lockable in position. A guide is removably attached to the distal end of the post and acts to guide the placement of the second plate during assembly, particularly assembly in situ during surgery. The guide is advantageously flexible, or at least more flexible than the post, so that the guide may be routed conveniently during surgery. The guide is removable from the distal end of the post after assembly, prior to closing the surgical site.

### BACKGROUND

The present invention generally relates to devices and methods for stabilizing vertebral members, and more particularly, to spinal implants that mount onto the spinous processes.

Vertebral members typically comprise a vertebral body, pedicles, laminae, and processes. The processes are projections that serve as connection points for the ligaments and tendons, and typically include the articular processes, transverse processes, and the spinous process. Intervertebral discs are located between adjacent vertebral bodies to permit flexion, extension, lateral bending, and rotation.

Various conditions may lead to damage of the intervertebral discs and/or the vertebral members. The damage may result from a variety of causes including a specific event such as trauma, a degenerative condition, a tumor, or infection. Damage to the intervertebral discs and vertebral members can lead to pain, neurological deficit, and/or loss of motion. One manner of correcting the damage involves mounting of a spinal implant onto the spinous processes, typically in association with a fixation process such as anterior lumbar interbody fusion (ALIF), posterior lumbar interbody fusion (PLIF), intertransverse lumbar interbody fusion (ILIF), and the like. See, for example, the spinal implant sold under the trade name CD HORIZON SPIRE™ by Medtronic Spinal and Biologics of Memphis, Tenn., and the devices described in U.S. Pat. Nos. 7,048,736 and 7,727,233. While these devices provide some solutions, they may not be ideal for some situations. As such, there remains a need for alternative spinal implants and related methods.

### SUMMARY

The present application is directed to implants for attaching to spinous processes and methods for attaching implants to spinous processes. In some embodiments, the implants include a first plate sized to extend along a first lateral side of the spinous processes, and a second plate sized to extend along a second lateral side of the spinous processes. The second plate includes a first bore that extends through the second plate between a medial surface that faces towards the spinous processes and an opposing outer surface. An elongated post extends along a post longitudinal axis from a proximal end attached to the first plate to a distal end positioned distally beyond the outer surface of the second plate away from the first plate. The post extends through the first bore. The first bore is sized relative to the post for the second plate to be movable along a length of the post and selectively locked in position therealong. A guide is distinct from the post and removably mounted to the distal end of the post. The guide extends distally from the post along a guide longitudinal axis from a guide proximal end to a guide distal end. The post has a first cross-sectional area normal the post longitudinal axis. The guide distal end has a second cross-sectional area normal to the guide longitudinal axis, with the first cross-sectional area larger than the second cross-sectional area. The second plate, post, and guide are collectively configured such that the guide proximal end may be inserted into the first bore, then the second plate slid over the guide and onto the post, such that the post extends through the first bore.

The second plate may include a second bore disposed generally orthogonal to the first bore and intersecting the first bore, and the implant may further include a locking member movable in the second bore to lock the relative position of the second plate along the post. The locking member may be a setscrew. At least one, and advantageously both, of the first and second plates may include projections on a medial face thereof that project toward the other of the first and second plates. The guide may advantageously be flexible. The post distal end may comprise a third bore disposed along the post longitudinal axis, with the guide proximal end grippingly disposed in the third bore. The third bore and the guide proximal end may have respective threads for threadably engaging with each other. The post may advantageously be pivotable relative to the first plate. The guide may be formed from a shape memory material. The post and guide may be configured such that the guide can be dismounted from the post by movement in a direction along the post longitudinal axis.

In some embodiments, the implants include a first plate sized to extend along a first lateral side of the spinous processes, and a second plate sized to extend along a second lateral side of the spinous processes. The second plate includes a first bore that extends through the second plate between a medial surface that faces towards the spinous processes and an opposing outer surface. An elongated post extends along a post longitudinal axis from a proximal end attached to the first plate to a distal end positioned distally beyond the outer surface of the second plate away from the first plate. The post extends through the first bore and the first bore is sized relative to the post for the second plate to be movable along a length of the post and selectively locked in position therealong. A guide is distinct from the post and removably mounted to the distal end of the post. The guide extends distally from the post along a guide longitudinal axis from a guide proximal section to a guide distal section, with a post intermediate section disposed therebetween. The post proximal section includes a taper section, with the taper section distally tapering inward toward the post longitudinal axis. The taper section has a first longitudinal length. The guide intermediate section extends for a second longitudinal length, with the second longitudinal length longer than the first longitudinal length. The guide intermediate section and distal section are both smaller in cross-section than the post distal end. The second plate, post, and guide are collectively configured such that the guide proximal section may be inserted into the first bore, then the second plate slid over the guide and onto the post, such that the post extends through the first bore.

The guide distal section may advantageously be more flexible than the post. The guide proximal section may further include a stub section disposed proximally relative to the taper section, with the stub section smaller in cross-section than the tapering section and the post and abutting the post. The second plate may include a second bore disposed generally orthogonal to the first bore and intersecting the first bore, and a locking member may be movable in the second bore to lock the relative position of the second plate along the post.

In some embodiments, a method of attaching an implant to spinous processes may include positioning a first plate on a first lateral side of the spinous processes and positioning a post that extends outward from the first plate through an interspinous space formed between the spinous processes. The method includes sliding a second plate onto the post by first sliding the second plate onto an elongate guide extending from a distal end of the post and thereafter positioning the second plate on a second lateral side of the spinous processes with the post extending through a bore in the second plate. The method includes pressing the first and second plates toward each other so that the plates bite into opposing sides of the spinous processes. The method includes securing the second plate at a selected longitudinal position on the post relative to the first plate. The method includes detaching the guide from the post.

The detaching the guide from the post may occur prior to the pressing the first and second plates toward each other so that the plates bite into opposing sides of the spinous processes. The method may include bending the elongate guide prior to the sliding the second plate onto the guide. The method may be such that detaching the guide from the post comprises moving the guide in a direction that is coincident with a longitudinal axis of the post. The guide may comprise a proximal section, a distal section, and an intervening intermediate section; wherein during the sliding the second plate onto the guide, the proximal section of the guide is mounted at a recess on a distal end of the post, and the intermediate section of the guide has a cross section smaller than a cross section of the post over which the second plate slides. The proximal section may include a taper section that tapers inward in a distal direction from a cross section that substantially matches the cross section of the post over which the second plate slides to the smaller cross section of the guide intermediate section, and the sliding the second plate onto the post may comprise aligning the plate with the post via the taper section of the guide.

The various aspects of the various embodiments may be used alone or in any combination, as is desired.

### BRIEF DESCRIPTION OF THE DRAWINGS

FIG. 1 shows a spinal implant according to one embodiment mounted to a spinal column.

FIG. 2 shows a perspective view of the spinal implant of FIG. 1 with the guide attached.

FIG. 3 shows a perspective view of the spinal implant of FIG. 1 with the guide detached.

FIG. 4 shows a top (posterior to anterior) view of the implant of FIG. 3.

FIG. 5 shows an alternative embodiment with a guide having a threaded stem.

FIG. 6 shows an implant during a first phase of the assembly process, with the guide attached and the second plate not yet mounted to the guide.

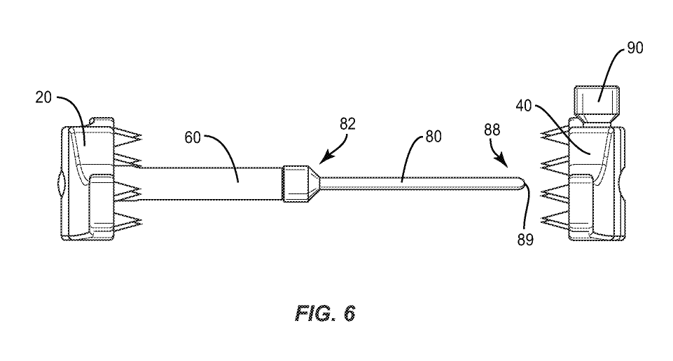

FIG. 7 shows the implant of FIG. 6 during a second phase of the assembly process, with the second plate slid onto the guide intermediate section.

FIG. 8 shows the implant of FIG. 6 during a third phase of the assembly process, with the second plate slid onto the guide proximal section.

FIG. 9 shows the implant of FIG. 6 during a fourth phase of the assembly process, with the second plate slid onto the post and the guide still attached.

FIG. 10 shows the implant of FIG. 6 during a fifth phase of the assembly process, with the second plate displaced toward the first plate and the fastener tightened to lock the second plate relative to the post, and the guide removed.

### DETAILED DESCRIPTION

In one embodiment, the present application is directed to a spinal implant with two plates that are connected together by a post. The implant is configured for each plate to be positioned on outer lateral sides of spinous processes with the post extending through the interspinous space. The second of the two plates includes a bore that receives the post, and that plate is movable along the length of the post to accommodate different anatomies such as for relatively wide or thin spinous processes, and selectively lockable in position. A guide is removably attached to the distal end of the post, and acts to guide the placement of the second plate during assembly, particularly assembly in situ during surgery. The guide is advantageously flexible, or at least more flexible than the post, so that the guide may be routed conveniently during surgery. The guide is removable from the distal end of the post after assembly, prior to closing the surgical site. As such, the spinal implant allows for an easier assembly process, particularly assembly in situ during surgery.

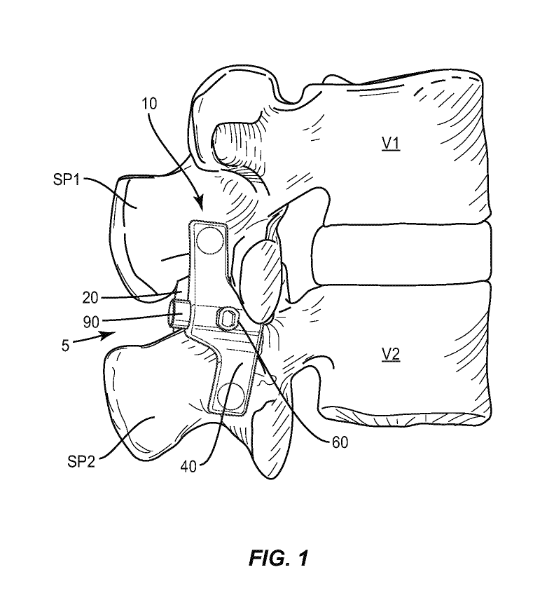

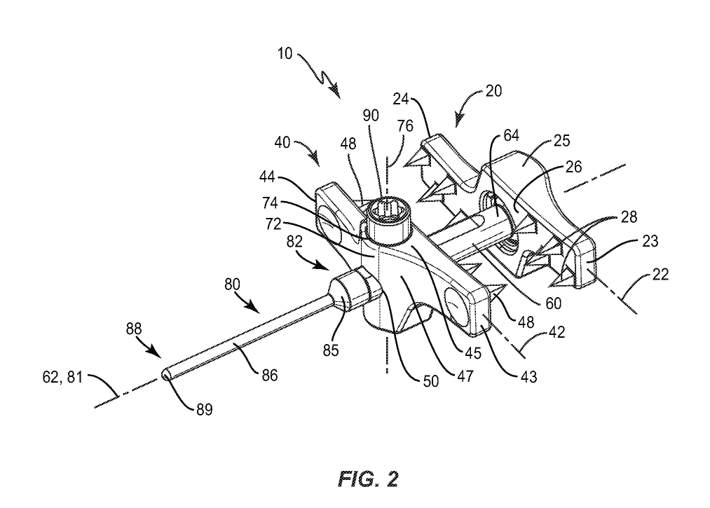

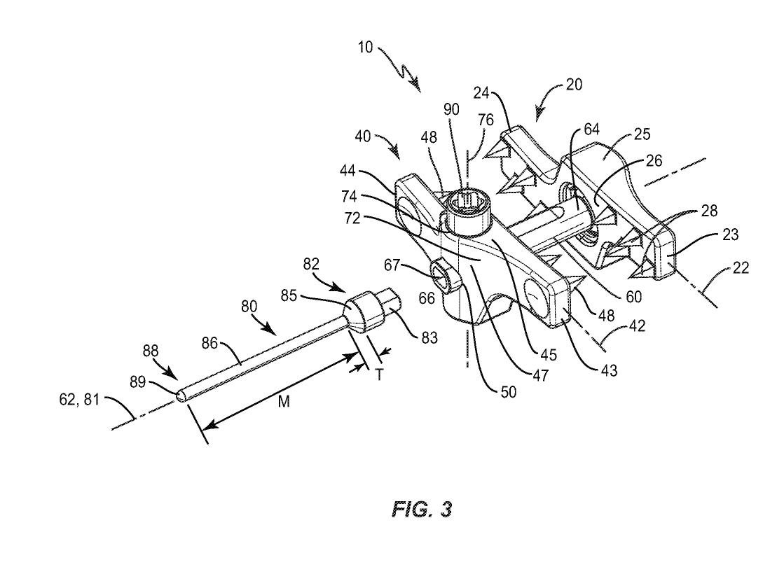

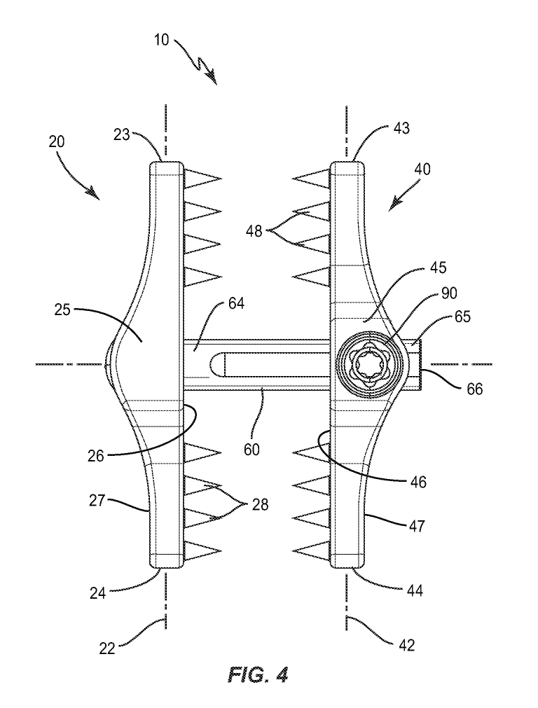

Referring to FIG. 1, a spinal implant according to one embodiment and generally designated 10 is shown clampingly mounted to the spinous process SP1 of a superior vertebra V1 and a spinous process SP2 of an inferior vertebra V2. A portion of the implant 10, in particular post 60, extends transversely through the interspinous space 5 between the two spinous processes SP1,SP2. The implant, shown more clearly in FIGS. 2-5, includes a first plate 20, a second plate 40, an interconnecting post 60, a removable guide 80, and a fastener 90.

Referring to FIGS. 2-4, the first plate 20 may be elongate along an associated longitudinal axis 22, with a superior end section 23, an inferior end section 24, and an intermediate section 25. If desired, the superior end section 23 and inferior end section 24 may be shifted in an anterior direction or a posterior direction so that the first plate has a somewhat Z-shape as shown, although this is optional and the first plate 20 may be generally rectilinear or any other suitable shape as is desired. The first plate 20 has a length sufficient to vertically span the interspinous gap 5 (interspinous space) between adjacent spinous processes while substantially overlapping the spinous processes SP1,SP2. The first plate 20 has a medial face 26 and an opposite lateral face 27. The medial face 26 includes a plurality of protrusions 28 that extend medially for biting into the corresponding spinous process SP1,SP2. Advantageously, the protrusions take the form of a plurality of sharp teeth. The teeth 28 may advantageously be disposed in two groups, one on the superior end section 23 and one on the inferior end section 24, with the intermediate section 25 being free of such teeth 28. The lateral face 27 may have suitable features, such as recesses or the like, for cooperating with installation and manipulation instrumentation. The tips of superior end 23 and inferior end 24 are advantageously generally rounded so as to minimize damage to surrounding tissue and for ease of installation.

The second plate 40 may be substantially similar to the first plate 20. For example, the second plate 40 may be elongate along an associated longitudinal axis 42, with a superior end section 43, an inferior end section 44, and an intermediate section 45. If desired, the second plate 40 may have a somewhat Z-shape similar to the first plate 20, or may be any other suitable shape as is desired. The second plate 40 advantageously has a length sufficient to vertically span the interspinous gap 5 while substantially overlapping the spinous processes SP1,SP2. The second plate 40 has a medial face 46 and an opposite lateral face 47, with the medial face 46 facing the medial face 26 of the first plate 20. The medial face 46 includes a plurality of protrusions 48 similar to teeth 28 for biting into the spinous processes SP1,SP2. The lateral face 47 may have suitable features, such as recesses or the like, for cooperating with installation and manipulation instrumentation. The tips of superior end section 43 and inferior end section 44 are advantageously generally rounded so as to minimize damage to surrounding tissue and for ease of installation. The intermediate section 45 may have a suitable boss 72 thereon, with a hole 74 having centerline 76 for receiving the fastener 90, as discussed below. The intermediate section 45 of the second plate 40 includes a bore 50 that extends from medial face 46 to lateral face 47, through the intermediate section 45. The bore 50 is sized to receive post 60, and therefore has a cross-section at least as large, and advantageously larger than post 60, described further below.

The post 60 may take the form of a round shaft that extends along a post longitudinal axis 62 from a post proximal section 64 proximate the first plate 20 to a post distal section 65 proximate the second plate 40. The post 60 has a length sufficient to extend laterally across the interspinous gap 5, through the medial face 46 of the second plate 40, and into engagement with the fastener 90. In some embodiments, the post proximal section 64 is mounted to the first plate 20 by any suitable means, such as welding or the like. Alternatively, the post 60 may be integrally formed with the first plate 20. Still further, the post 60 may be pivotally attached to the first plate 20. For example, the post 60 may be pivotally attached either for monoaxial or polyaxial movement relative to the first plate 20 about one or more pivot axes, such as about a pivot axis perpendicular to the post axis 62 and parallel to medial face 26. Examples of suitable pivoting structures are shown in U.S. Pat. Nos. 7,048,736 and/or 7,727,233. The post 60 may include flats or other features (not shown) for engaging with the fastener 90. The post 60 may advantageously be rigid, and generally solid rather than hollow. The distal section 65 terminates at a distal tip 66. A longitudinally extending recess 67 extends inward from the distal tip 66, generally along the post axis 62. The recess 67 may be threaded in some embodiments, but is advantageously smooth sided, with a slight inward taper in the proximal direction. The post distal section 65 may have a cylindrical cross-sectional shape, or may have other convenient cross-sectional shapes, such as rectangular, hexagonal, oval, and the like.

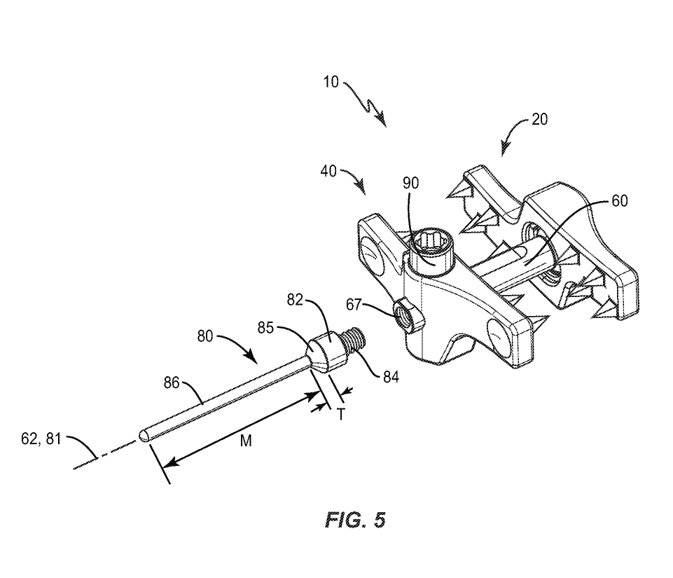

The guide 80 is removably mounted at the post distal section 65, and acts as a distinct and removable temporary extension of the post 60. The guide 80 may take a variety of forms, such as those shown in FIGS. 2 and 5. The guide 80 is an elongate member that extends along a guide longitudinal axis 81 from a proximal section 82 mounted to the post 60, to a guide intermediate section 86, and then to a guide distal section 88 disposed in spaced relation to the post 60. As can be appreciated, the guide intermediate section 86 is disposed between the proximal section 82 and the distal section 88. The proximal section 82 advantageously includes a stem or stub section 83 and a taper section 85. The stub section 83 is sized and configured for releasable gripping connection in the recess 67 of post 60. In some embodiments, the stub section 83 is threaded. In other embodiments, the stub section 83 has a generally smooth exterior that may be cylindrical or any other shape corresponding to the shape of the recess 67. The stub section 83 may include a short end taper to aid in insertion of the stub section 83 into recess 67 in a direction along post axis 62. The stub section 83 advantageously has a slight press fit with recess 67 when mated to post 60, so that stub section 83 (and therefore guide 80) may be readily removed from post 60 when desired, but the stub section 83 will not inadvertently come loose during normal surgical handling. As can be appreciated, the stub section 83 is smaller in cross section than the post 60. The taper section 85, on the other hand, advantageously starts out with a cross section that matches the cross section of the post 60 in size and shape, and then tapers inwardly, in the distal direction, toward the guide axis 81. The inward taper ends at the junction of the proximal section 82 with the guide intermediate section 86. The proximal section 82 may optionally include a central section between the stem section 83 and the taper section 85, which advantageously has a constant cross-section corresponding in shape to the post 60. The guide intermediate section 86 advantageously takes the form of a cylindrical section that is relatively smaller in cross section than the post 60. The intermediate section 86 is advantageously longer than the taper section 85. For example the length L2 of the intermediate section 86 may be about three to about ten times longer than the length L1 of the taper section 85. The guide 80 distal section includes the distal tip 89 of the guide 80, which is advantageously rounded. The guide 80 may be somewhat stiff, but is advantageously more flexible than the post 60 so that the guide 80 may be bent as desired, and approximately hold the bent shape, during the surgical procedure. Alternatively, the guide 80 may be readily flexible, such as being made from a flexible polymer.

The fastener or locking member 90 may take the form of a simple setscrew, optionally with tapered tip, that is sized to threadably engage hole 74 in second plate 40. When tightened, the locking member 90 presses against post 60 to lock the relative distance between the plates 20,40. Of course, other forms of fasteners, such as concentrically barbed posts, quarter-turn fasteners, and the like, may alternatively be used.

Clamping plates 20,40 to the spinous processes SP1,SP2 helps maintain the alignment and spacing of the spinous processes SP1,SP2 while also providing resistance to spinal extension and flexion. Thus, engagement of plates 20,40 to the spinous processes SP1,SP2 resists movement of the spinous processes SP1,SP2 toward and away from one another as a result of spinal extension and flexion, respectively, or as a result of any other movement or condition.

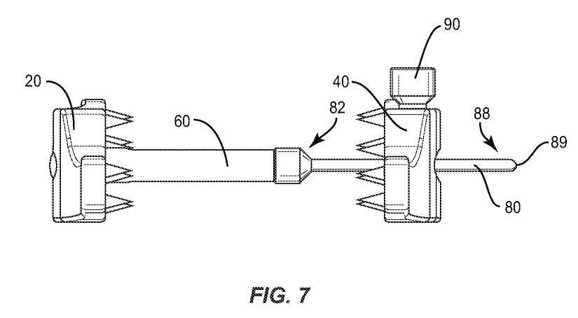

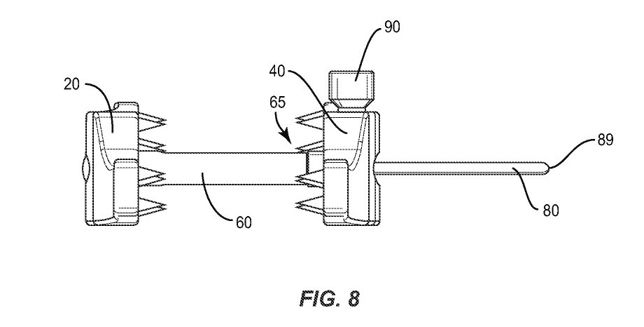

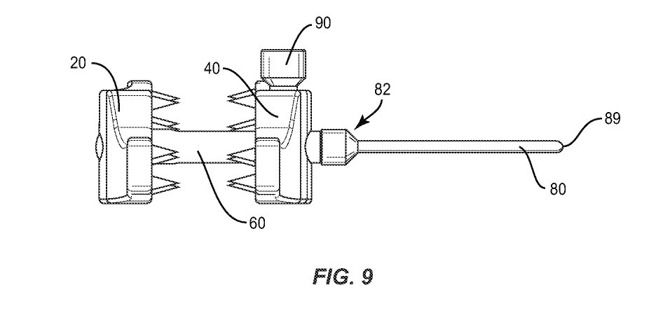

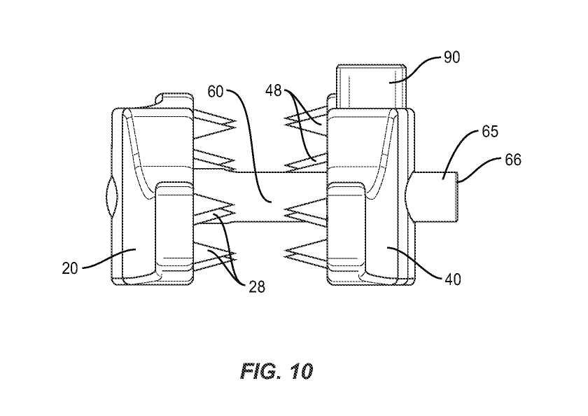

In use, the device 10 can be implanted for posterior spinal stabilization as a stand-alone procedure or in conjunction with other procedures. The device 10 can be positioned through a small posterior incision in the patient of sufficient size to admit the device and instrumentation. Following the incision, muscle is moved aside if and as needed for placement of the device 10. The spinous processes SP1,SP2 are optionally distracted using suitable instrumentation known in the art, and the first plate 20 is implanted such that the superior end section 23 extends on a first lateral side of spinous process SP1, inferior end section 24 extends on the first side of spinous process SP2, and post 60 extends through the interspinous space 5 generally normal to the sagittal plane defined by the spinous processes SP1,SP2. Note that the guide 80 is advantageously mated to the post 60 prior to inserting the first plate 20, so that the guide 80 acts as an extension of the post 60. Due to the more flexible nature of the guide 80, the distal tip 89 of the guide 80 advantageously extends posteriorly away from the surgical site toward the surgeon, when the first plate 20 is installed. With the first plate 20 in position, the distal tip 66 of post 60 extends laterally beyond the spinous processes SP1,SP2 on the lateral side opposite first plate 20, and the guide 80 extends beyond that. The second plate 40 may then be added by inserting the guide tip 89 through the bore 50, and sliding the second plate 40 down the guide 80 toward the first plate 20. As the second plate 40 is slid toward the post 60, the second plate 40 moves over the guide distal section 88, then guide intermediate section 86 (see FIG. 7), then over the guide proximal section 82 (see FIG. 8) and onto post 60 (see FIG. 9). As can be appreciated, the post taper section 85 helps position the second plate 40 properly so that the post 60 may easily enter the bore 50 when the second plate 40 is slid onto the post 60. When the second plate 40 is advanced sufficiently so that the post distal tip 66 is extending distally beyond the lateral face 47 of second plate 40 (i.e., the post 60 extends entirely through bore 50), the plates 20,40 are pushed toward one another with a compression instrument or manually, to move (e.g., slide) the second plate 40 along the post 60 toward the first plate 20 from a first position farther from the first plate 20 to a second position closer to the first plate 20. This movement causes the plates 20,40 to clamp the spinous processes SP1,SP2, with the spikes 28,48 biting into the bony material of the spinous processes SP1,SP2. Locking member 90 is then tightened onto post 60 using an appropriate instrument to lock the relative positions of the plates 20,40. See FIG. 10. If desired, locking member 90 may be provided with a break-off portion (not shown) that provides an indication when sufficient torque is applied. Note that the second plate 40 is advantageously positionable along post 60 at an infinite number of positions, as the second plate 40 can conceptually slide to any number of positions along post 60 and be locked in the selected position by fastener 90. Once the plates 20,40 are locked in position, the guide 80 may be removed from the post 60 by pulling the guide 80 distally in a direction along the post axis 62, so as to remove the stem section 83 from recess 67, thereby disconnecting the guide 80 from post 60.

The above description has been in the context of removing the guide 80 from the post 60 after locking the second plate 40 in position. However, the guide 80 may be removed from the post 60 at any point in the process after the second plate 40 has been guided onto the post 60 and prior to closing the surgical site, including immediately prior to locking the second plate 40 in position or prior to pressing the plates 20,40 toward each other. Further, while the above description has been in the context of an in-situ assembly of the first and second plates 20,40, in some embodiments, the device 10 may be inserted in an already-assembled condition, with the second plate 40 disposed on the post 60, with the device being either assembled by the manufacturer or by medical personnel.

As will be appreciated, the first plate 20, second plate 40, post 60, guide 80, and fastener 90 may each be made from any suitable biocompatible rigid materials such as titanium and its alloys, stainless steel, cobalt chrome, ceramics, relatively rigid polymers like carbon reinforced polyetheretherketone (PEEK), or the like, known in the art. Advantageously, the guide is made from a material having a lower modulus of elasticity than the post 60, such as a cobalt chrome post 60, and a Nitinol or silicone guide 80. As can be appreciated, the first plate 20, second plate 40, guide 70, and fastener 90 are advantageously distinct (i.e., separate) pieces from each other that are joined together during assembly.

In some embodiments, a sleeve (not shown) may be disposed on post 60 to provide additional support of the vertebrae to maintain or provide post-operative distraction between the spinous processes SP1,SP2. The sleeve may be osteoconductive if desired. For more information on sleeves, see U.S. Pat. No. 7,727,233. The sleeve may be slid over guide 80 toward post 60 in a similar fashion as described above, prior to inserting guide 80 into bore 50 of second plate 40.

While FIG. 1 shows an implant applied to vertebra L-4 and L-5, the implant device can be implanted on spinous processes at other levels. Levels up to T-3 may be appropriate sites. Also, plates 20,40 bridging more than one level may also be considered, optionally with multiple posts 60 and guides 80 disposed at suitable intervals.

The implant 10 may be used during surgical procedures on living patients. The implant may also be used in a non-living situation, such as within a cadaver, model, and the like. The non-living situation may be for one or more of testing, training, and demonstration purposes.

All U.S. patents, patent application publications, and applications mentioned above are hereby incorporated herein by reference in their entirety.

The present invention may, of course, be carried out in other ways than those specifically set forth herein without departing from essential characteristics of the invention. The present embodiments are to be considered in all respects as illustrative and not restrictive, and all changes coming within the meaning and equivalency range of the appended claims are intended to be embraced therein.

What is claimed is:

1. An implant for attaching to adjacent spinous processes comprising:
a first plate sized to extend along a first lateral side of the spinous processes;
a second plate sized to extend along a second lateral side of the spinous processes, the second plate including a first bore that extends through the second plate between a medial surface that faces towards the spinous processes and an opposing outer surface;
an elongated post extends along a post longitudinal axis from a proximal end attached to the first plate to a distal end positioned distally beyond the outer surface of the second plate away from the first plate; the post extending through the first bore;
the first bore sized relative to the post for the second plate to be movable along a length of the post and selectively locked in position therealong;
a guide distinct from the post and removably mounted to the distal end of the post; the guide extending distally from the post along a guide longitudinal axis from a guide proximal end to a guide distal end;
wherein the post has a first cross-sectional area normal the post longitudinal axis; wherein the guide distal end has a second cross-sectional area normal to the guide longitudinal axis; wherein the first cross-sectional area is larger than the second cross-sectional area;
wherein the second plate, post, and guide are collectively configured such that the guide proximal end may be inserted into the first bore, then the second plate slid over the guide and onto the post, such that the post extends through the first bore.

2. The implant of claim 1 wherein the second plate includes a second bore disposed generally orthogonal to the first bore and intersecting the first bore; wherein the implant further includes a locking member movable in the second bore to lock the relative position of the second plate along the post.

3. The implant of claim 1 wherein the locking member is a setscrew.

4. The implant of claim 1 wherein at least one of the first and second plates includes projections on a medial face thereof that project toward the other of the first and second plates.

5. The implant of claim 1 wherein the guide is flexible.

6. The implant of claim 1 wherein the post distal end comprises a third bore disposed along the post longitudinal axis; wherein the guide proximal end is grippingly disposed in the third bore.

7. The implant of claim 6 wherein the third bore and the guide proximal end have respective threads for threadably engaging with each other.

8. The implant of claim 1 wherein the post is pivotable relative to the first plate.

9. The implant of claim 1 wherein the guide is formed from a shape memory material.

10. The implant of claim 10 wherein the post and guide are configured such that the guide can be dismounted from the post by movement in a direction along the post longitudinal axis.

11. An implant for attaching to adjacent spinous processes comprising:
a first plate sized to extend along a first lateral side of the spinous processes;
a second plate sized to extend along a second lateral side of the spinous processes, the second plate including a first bore that extends through the second plate between a medial surface that faces towards the spinous processes and an opposing outer surface;
an elongated post extends along a post longitudinal axis from a proximal end attached to the first plate to a distal end positioned distally beyond the outer surface of the second plate away from the first plate; the post extending through the first bore;
the first bore sized relative to the post for the second plate to be movable along a length of the post and selectively locked in position therealong;
a guide distinct from the post and removably mounted to the distal end of the post; the guide extending distally from the post along a guide longitudinal axis from a guide proximal section to a guide distal section, with a post intermediate section disposed therebetween;
wherein the post proximal section includes a taper section; the taper section distally tapering inward toward the post longitudinal axis; wherein the taper section has a first longitudinal length;
wherein the guide intermediate section extends for a second longitudinal length, the second longitudinal length longer than the first longitudinal length;
wherein the guide intermediate section and distal section are both smaller in cross-section than the post distal end;
wherein the second plate, post, and guide are collectively configured such that the guide proximal section may be inserted into the first bore, then the second plate slid over the guide and onto the post, such that the post extends through the first bore.

12. The implant of claim 11 wherein the guide distal section is more flexible than the post.

13. The implant of claim 11 wherein the guide proximal section further includes a stub section disposed proximally relative to the taper section; the stub section smaller in cross-section than the tapering section and the post; the stub section abutting the post.

14. The implant of claim 11 wherein the second plate includes a second bore disposed generally orthogonal to the first bore and intersecting the first bore; wherein the implant further includes a locking member movable in the second bore to lock the relative position of the second plate along the post.

15. A method of attaching an implant to spinous processes comprising:
positioning a first plate on a first lateral side of the spinous processes;
positioning a post that extends outward from the first plate through an interspinous space formed between the spinous processes;
sliding a second plate onto the post by first sliding the second plate onto an elongate guide extending from a distal end of the post and thereafter positioning the second plate on a second lateral side of the spinous processes with the post extending through a bore in the second plate;
pressing the first and second plates toward each other so that the plates bite into opposing sides of the spinous processes;
securing the second plate at a selected longitudinal position on the post relative to the first plate;
detaching the guide from the post.

16. The method of claim 15 wherein the detaching the guide from the post occurs prior to the pressing the first and second plates toward each other so that the plates bite into opposing sides of the spinous processes.

17. The method of claim 15 further comprising bending the elongate guide prior to the sliding the second plate onto the guide.

18. The method of claim 15 wherein the detaching the guide from the post comprises moving the guide in a direction that is coincident with a longitudinal axis of the post.

19. The method of claim 15 wherein the guide comprises a proximal section, a distal section, and an intervening intermediate section; wherein during the sliding the second plate onto the guide, the proximal section of the guide is mounted at a recess on a distal end of the post; wherein the intermediate section of the guide has a cross section smaller than a cross section of the post over which the second plate slides.

20. The method of claim 19 wherein the proximal section includes a taper section that tapers inward in a distal direction from a cross section that substantially matches the cross section of the post over which the second plate slides to the smaller cross section of the guide intermediate section; wherein the sliding the second plate onto the post comprises aligning the plate with the post via the taper section of the guide.
